(19) United States
(12) Patent Application Publication
(10) Pub. No.: US 2022/0062660 A1
(43) Pub. Date: Mar. 3, 2022

(54) ULTRASONIC SYSTEM FOR SKIN-TIGHTENING OR BODY-SHAPING TREATMENT

(71) Applicants: Ines VERNER RASHKOVSKY, Savyon (IL); Yoram ESHEL, Netanya (IL)

(72) Inventors: Ines VERNER RASHKOVSKY, Savyon (IL); Yoram ESHEL, Netanya (IL)

(21) Appl. No.: 17/312,805

(22) PCT Filed: Dec. 11, 2019
(86) PCT No.: PCT/IL2019/051357

§ 371 Date: Jun. 10, 2021

Related U.S. Application Data
(60) Provisional application No. 62/777,940, filed on Dec. 11, 2018.

(51) Int. Cl. A61N 7/00; A61B 18/20; A61B 8/08

(52) U.S. Cl. CPC A61N 7/00; A61B 18/203; A61B 8/0858; A61B 2018/00452; A61N 2007/0034; A61N 2007/0078; A61N 2007/0095; A61N 2007/0008

(57) ABSTRACT

A system for providing an ultrasonic skin-tightening treatment is disclosed. The system comprises a) a flexible sleeve wrapped around a treatment volume of a patient; b) one or more treatment panels, arranged on an inside wall of the sleeve, provide ultrasound energy and cooling to the treatment volume; and c) a control module controls ultrasonic and cooling parameters independently for each treatment panel. The system enables hands-free treatment over an entire treatment volume, with localized variations of the parameters, according to treatment requirements, under each treatment panel. The ultrasound and cooling parameters may be varied to select treatment of a particular depth below the skin surface.

### FIELD OF THE INVENTION

The invention is in the field of skin tightening systems and methods, in particular to those applying ultrasonic energy to the skin and sub-cutaneous tissue thereof.

### BACKGROUND

Non-invasive body contouring and tissue tightening by energy-based devices (EBDs) have gained popularity in recent years and form now the largest segment in the aesthetic market.

US 2001/0014819 A1 discloses devices, methods, and systems for shrinking of collagenated tissues, particularly for treating urinary incontinence in a noninvasive manner by directing energy to a patient's own support tissues. This energy heats fascia and other collagenated support tissues, causing them to contract. The energy can be applied intermittently, often between a pair of large plate electrodes having cooled flat electrode surfaces, the electrodes optionally being supported by a clamp structure. Such cooled plate electrodes are capable of directing electrical energy through an intermediate tissue and into fascia while the cooled electrode surface prevents injury to the intermediate tissue, particularly where the electrode surfaces are cooled before, during, and after an intermittent heating cycle. Ideally, the plate electrode comprises an electrode array including discrete electrode surface segments so that the current flux can be varied to selectively target the fascia.

US 2018/0130457 discloses an ultrasound array comprising a plurality of ultrasound transducer elements on a carrier, the carrier further carrying an actuator arrangement of a material having an adjustable shape in response to an electromagnetic stimulus. e.g. an electro active polymer or optically responsive polymer, wherein tile material is arranged to change the orientation of said ultrasound transducer elements in response to said stimulus. This facilitates configurable beam shaping and/or body contour matching with the ultrasound array. An ultrasound system composing such an ultrasound array is also disclosed.

U.S. Pat. No. 10,292,859 discloses a cooling device for removing heat from subcutaneous lipid-rich cells of a subject having skin. The cooling device includes a plurality of cooling elements movable relative to each other to conform to the contours of the subject's skin. The cooling elements have a plurality of controllable thermoelectric coolers. The cooling elements can be controlled to provide a time-varying cooling profile in a predetermined sequence, can be controlled to provide a spatial cooling profile in a selected pattern, or can be adjusted to maintain constant process parameters, or can be controlled to provide a combination thereof.

U.S. Pat. No. 8,915,948 discloses a method and apparatus for treating tissue in a region at depth by applying optical radiation thereto of a wavelength able to reach the depth of the region and of a selected relatively low power for a duration sufficient for the radiation to effect the desired treatment while concurrently cooling tissue above the selected region to protect such tissue. Treatment may be enhanced by applying mechanical, acoustic or electrical stimulation to the region.

U.S. Pat. No. 10,265,550 discloses a probe for ultrasound treatment of skin laxity. Systems and methods can include ultrasound imaging of the region of interest for localization of the treatment area, delivering ultrasound energy at a depth and pattern to achieve the desired therapeutic effects, and/or monitoring the treatment area to assess the results and/or provide feedback. In an embodiment, a treatment system and method can be configured for producing arrays of sub-millimeter and larger zones of thermal ablation to treat the epidermal, superficial dermal, mid-dermal or deep dermal components of tissue.

U.S. Pat. No. 9,108,037 B2 discloses a tissue ablation system that facilitates lesioning deep tissue while preventing damage to superficial tissue and includes a probe having a distal end portion, at least one transducer carried on the distal end portion, and at least one acoustically transparent heat removal element thermally coupled to a target tissue within the beam path of the transducer. The transducer delivers acoustic energy to the tissue through the heat removal element in order to ablate the tissue; the heat removal element removes sufficient thermal energy from the tissue volume to prevent thermal necrosis in superficial tissue. The heat removal element may be a heat sink or a convective element. An optional temperature sensor provides advisory data to a practitioner and/or is coupled to a feedback control system operable to control delivery of acoustic energy to the tissue and/or a rate of thermal energy removal therefrom.

Novel and inventive features of the present invention advance the field of systems for skin-tightening and body shaping, as described below.

### SUMMARY

The main emphasis in the development of tissue tightening systems has been on the dermis. The present invention arises in part from an understanding that not only the dermis but also the subcutaneous fat tissue has to be tightened to achieve optimal tightening and contouring results.

Tissue tightening can be achieved by heating the dermis and the fibroseptal network in the subcutaneous fat tissue. This heating leads to shrinkage of collagen and to stimulation of new collagen production and thus tissue tightening. Heating the connective tissue in the dermis and in the subcutaneous fat to 40-55° C. induces collagen shrinkage and partially reversible damage with stimulation of new collagen production. Heating to above 55° C. causes destruction of chemical bonds, immediate collagen denaturation and immediate and delayed skin tightening. Tightening of skin and underlying tissue leads to tighter, younger-looking tissue.

Moreover it has been shown in multiple in-vitro studies (e.g., “Hyperthermic injury to adipocyte cells by selective heating of subcutaneous fat with a novel radiofrequency device: Feasibility studies”; Franco W et al.; Lasers Surg Med 2010; 42(5):361-370) that adipocytes are heat sensitive and that heating fat tissue for a certain time to up to 50° C. induces fat cell apoptosis or delayed adipocyte cell death. Heating fat for three minutes at 45° C. induces apoptosis in 60% of cells; heating for 1 minute at 50° C. induces apoptosis in 80% of cells; and 15 minutes at 43-45° C. leads to 80% apoptosis. This controlled heating later leads to a reduction of the subcutaneous fat layer together with new collagen production and thus an improved body shape.

Up to now, little to no attention has been paid to the superficial fascia (SF). SF passes as a sheet of fibrous connective tissue composed of extracellular matrix (ECM) through the subcutaneous fat tissue (SFT) and separates the superficial fat layer from the deep fat layer. It holds together the structures underneath and is connected to the dermis by a fibrous septal connective tissue network that envelopes clusters of adipocyte cells. The SF is present nearly everywhere in the body; it is 1-3 mm thick and is at a depth between 3.5 mm and 2 mm beneath the skin's outside surface, the depth depending on body area, sex, age, and BMI.

The superficial fascia, together with the fibrous septa and the dermis, loosen with age due to collagen fragmentation and matrix breakdown. This leads to tissue sagging, wrinkles, uneven skin surface and cellulite.

To be able to achieve the most effective non-invasive body contouring, it is important therefore to address all layers involved in the pathology of tissue. Not only the skin and the superficial subcutaneous fat tissue with its fibrous septa should be treated, but also the fibrous superficial fascia underneath.

Multiple EBDs on the market are for body contouring. Some are radio-frequency or ultrasound based devices and exert their effect on the tissue by heating. Other devices cool the fat tissue and induce fat cell death and thus circumference reduction by cooling.

Most existing devices either tighten the skin (mainly RF- and ultrasound-based devices) or reduce the circumference (cooling devices, ultrasound, RF, or laser). However, using generally available devices it is not possible to achieve both tightening and body contouring in the same device and thus multiple devices and combinations of different treatments are needed to achieve the desired results.

For some of the devices a well-trained technician and multiple and long treatments are needed to achieve the desired results. As large areas of the body need to be covered, the treatment may be very long (lasting up to a few hours) and results may depend on the professionalism of the technician sliding a movable energy source, e.g. a wand, over the skin. Thus, these treatments are expensive (taking into account treatment time and consumables) and results may be variable and vary greatly from clinic to clinic.

Lately, hands free devices—mostly cooling devices and some heating devices—for circumference reduction have taken up a large segment of the market. Their results are easier to reproduce than for the hand-moved ones, as their use is less technician dependent.

Most technologies do not provide visualization of tissue before or during treatment, so it is not exactly clear at which depth the treatment should be performed (e.g. the superficial fascia depth is different from person to person) and which exactly what layers have been treated. Thus many technologies are not effective as they do not treat tissue at the correct depth. A few technologies do have visualization by ultrasound of the treated area (as disclosed in U.S. Pat. No. 10,265,550) but due to limited energy penetration depth cannot reach the needed depth to treat superficial fascia.

There is a need for a tissue tightening and body-contouring device; which diagnoses the treatment layer at the correct depth; which addresses both circumference reduction and tissue tightening in one device; which provides the operator with visualization of the treatment; and which is easy to operate, is hands free, and can offer marked results after only one or two treatment sessions.

The suggested invention offers a solution that is efficacious for both skin laxity (at treatment depths of 3-20 mm) and fat deposits (at treatment depths of 10-30 mm). It offers a hands-free, thermal based solution for heating the subcutaneous fat and fascia while keeping the epidermis cooled with a temperature below the level of causing damage.

It is therefore within the scope of the invention to provide a system for ultrasonic skin-tightening and body shaping treatment, comprising

a. a sleeve configured for fixedly wrapping around an organ of a mammalian body; the organ comprising a treated volume of a layer of tissue(s) underneath an area of skin surface of the organ;

b. an arrangement of one or more treatment panels disposed on an inside surface of the sleeve, each the treatment panel comprising
i. an ultrasonic element, configured to provide ultrasound waves to a portion of the treated volume underneath the treatment panel;
ii. a cooling plate, configured to remove heat from the treated volume portion; and

c. a control module, in electrical connection with the treatment panels, configured to receive temperature outputs of the temperature sensors and to control the ultrasonic elements—including intensity, frequency, and/or duty cycle of the ultrasound waves—and to control the cooling plate temperature;

wherein the control module is further configured to control the ultrasonic elements and the cooling plates of each the treatment panel independently, thereby enabling hands-free treatment, with localized variations in the controls as needed, throughout the treatment volume.

It is further within the scope of the invention to provide the abovementioned system, wherein one or more of the treatment panels comprises a temperature sensor configured to monitor temperature on an outside surface of the treated volume portion.

It is further within the scope of the invention to provide any of the abovementioned systems, wherein the controller is further configured to receive one or more of preliminary inputs from the user according to anatomical area and size of patient.

It is further within the scope of the invention to provide any of the abovementioned systems, wherein the sleeve comprises a wrap-around cuff or a closed elastic loop.

It is further within the scope of the invention to provide any of the abovementioned systems, wherein the sleeve is configured for wrapping around one or more of an arm, the neck, the abdomen, the back, a thigh, and the face.

It is further within the scope of the invention to provide any of the abovementioned systems, wherein the treatment panels are arranged on the sleeve inside surface in one dimension, in two dimensions, or any combination thereof.

It is further within the scope of the invention to provide any of the abovementioned systems, wherein the treatment panels cover a portion of skin underneath the sleeve.

It is further within the scope of the invention to provide any of the abovementioned systems, wherein the control module is configurable to disable one or more of the treatment panels during any time interval of the treatment.

It is further within the scope of the invention to provide any of the abovementioned systems, wherein the control module is further configured to change of ultrasound parameters to change from skin tightening to fat destruction.

It is further within the scope of the invention to provide any of the abovementioned systems, further comprising a tether providing electrical connections to the ultrasonic elements and the cooling plate from the control module.

It is further within the scope of the invention to provide any of the abovementioned systems, wherein a treatment depth, in a portion of the treatment volume underneath one or more of the treatment panels, is controlled by varying one or more of the intensity, frequency, and temperature.

It is further within the scope of the invention to provide any of the abovementioned systems, wherein the system is configured for treatment of skin laxity, wherein the depth is 3-20 mm, and of fat deposits, wherein the depth is 10-30 mm.

It is further within the scope of the invention to provide any of the abovementioned systems, further comprising ultrasound imaging transducers, the system further configured to

a. acquire real-time ultrasound images of the treatment volume before, during, and/or after the treatment;

b. analyze the images during treatment to determine cumulative effects of the treatment at varying depths of the treatment volume; and

c. adjust the treatment parameters in real time as a function of the cumulative effects.

It is further within the scope of the invention to provide the previous system, wherein the control module is configured to employ a neural network algorithm to compute the depth indication.

It is further within the scope of the invention to provide any of the abovementioned systems including ultrasound image transducers, wherein the system is further configured to determine a depth of treatment prior to the treatment.

It is further within the scope of the invention to provide any of the abovementioned systems including ultrasound image transducers, wherein the control module comprises a user interface that displays the real-time image and/or the depth indication.

It is further within the scope of the invention to provide a method for providing real-time in-treatment depth indications from ultrasound images of a treatment volume, comprising steps of

a. obtaining any of the abovementioned systems;

b. acquiring training images taken during treatments by the system;

c. annotating the training images with observed depths of treatment;

d. processing an aggregation of the annotated images to develop a neural network algorithm for indicating treatment depth as a function of an ultrasound image 520;

e. deploying the neural network algorithm to indicate a treatment depth in a depth-monitoring image taken during a treatment 525.

### BRIEF DESCRIPTION OF THE DRAWINGS

Examples illustrative of embodiments of the disclosure are described below with reference to figures attached hereto. Dimensions of components and features shown in the figures are generally chosen for convenience and clarity of presentation and are not necessarily shown to scale. Many of the figures presented are in the form of schematic illustrations and, as such, certain elements may be drawn greatly simplified or not-to-scale, for illustrative clarity. The figures are not intended to be production drawings.

FIGS. 1A-1D illustrate a patient-attached apparatus of a system for skin-tightening and body shaping, according to some embodiments of the invention.

FIG. 2 illustrates a patient-attached apparatus of a system for skin-tightening and body shaping, with four treatment panels inside of a sleeve fixedly tightened around an upper arm of a patient, according to some embodiments of the invention.

FIG. 3 illustrates the thermal effect of a treatment panel on a treatment volume, according to some embodiments of the invention.

FIG. 4 illustrates construction of a treatment panel, according to some embodiments of the invention.

FIG. 5 illustrates a patient-attached apparatus with ultrasound imaging transducers for acquiring images of a treatment volume in real time during a skin-tightening treatment, according to some embodiments of the invention.

FIG. 6 shows steps of a method for providing real-time in-treatment imaging of a treatment volume, according to some embodiments of the invention.

FIG. 7 shows a functional block diagram of a system for skin tightening and body shaping, according to some embodiments of the invention.

FIG. 8 shows a functional block diagrams of a system for skin tightening and body shaping with ultrasound imaging feedback, according to some embodiments of the invention.

FIGS. 9-18 show features of experiments supporting operability of the invention and their results.

### DETAILED DESCRIPTION

The present invention provides a novel system for skin tightening as described herein in detail.

Although various features of the disclosure may be described in the context of a single embodiment, the features may also be provided separately or in any suitable combination. Conversely, although the disclosure may be described herein in the context of separate embodiments for clarity, the disclosure may also be implemented in a single embodiment. Furthermore, it should be understood that the disclosure can be carried out or practiced in various ways, and that the disclosure can be implemented in embodiments other than the exemplary ones described herein. The descriptions, examples, and materials presented in herein should not be construed as limiting, but rather as illustrative.

The terms “treated volume” and “treatment volume” refer to a layer of tissue(s) underneath an area of skin surface that is to be treated with ultrasound.

The term “treatment volume portion” refers to some portion of the treatment volume, comprising tissue(s) underneath a particular area of the skin surface and/or at a particular depth beneath the skin surface.

The term “treatment surface” refers to the most superficial layer of a treatment volume, namely the epidermis. It may also refer to the portion of the epidermis on which a treatment panel of the invention is placed during treatment.

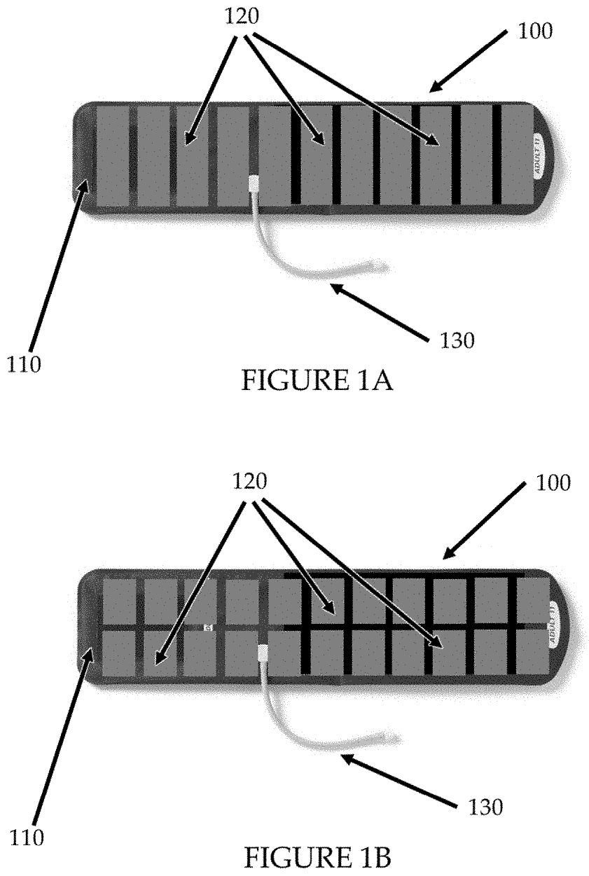

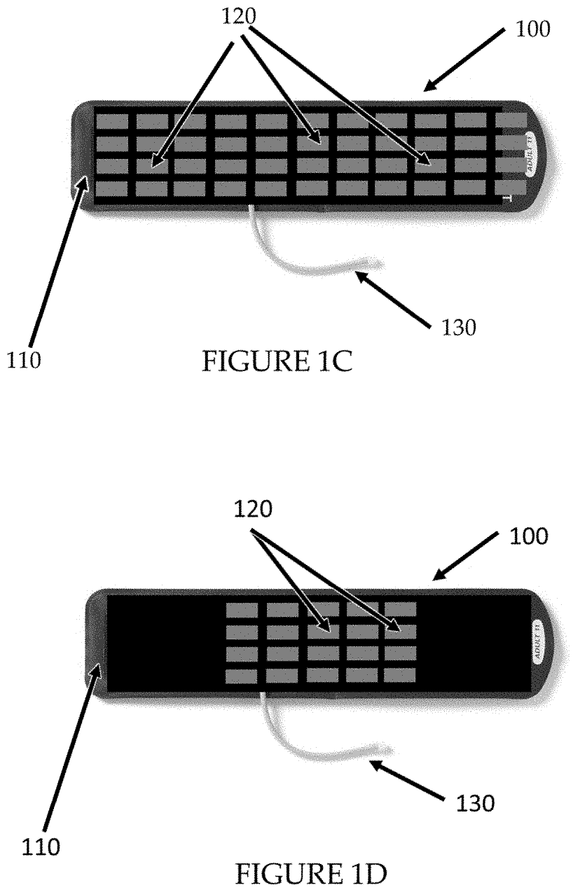

Reference is now made to FIG. 1A, illustrating a patient-attached apparatus 100 of a skin tightening system, according to some embodiments of the invention.

Apparatus 100 comprises a flexible or elastic sleeve 110, which can be a wrap-around cuff (similar to one used for a blood-pressure measurement apparatus). The wrap-around cuff may be secured by Velcro pads, snaps, laces, or adhesive tape. Alternatively, sleeve 110 can be a closed elastic loop or any flexible wrapping that can be fixedly wrapped around a treated volume of a patient. Sleeve 110 may be configured for wrapping around a treatment volume comprising any of one or more parts of the body, such as an arm, the neck, the abdomen, the back, a thigh, and any other anatomical areas that can be wrapped around. In some embodiments, a flexible mask is used for treatment of the face.

One or more treatment panels 120 are fixedly arranged on the surface of sleeve 110. Treatment panels 120 provide ultrasonic energy and heat removal (cooling) to the treatment volume, as further described herein. Treatment panels 120 face the inside of the sleeve 110, so as to provide ultrasonic and cooling treatment to the treatment volume. Treatment panels 120 may be secured to the inside of the sleeve 110. Alternatively, or in addition, treatment panels 120 may be secured to edges of openings in sleeve, for example, by stitching or fasteners.

As an alternative to a panel structure, one or more of the treatment elements providing ultrasound and cooling functions may have a cylindrical cross section.

A tether 130 connects treatment panels 120 to a control module (not shown). The control module provides driving power and signals to treatment panels 120. Alternatively, in some embodiments patient-connected apparatus 100 is wirelessly connected to the control module, whereby a power source and driving electronics are within patient-connected apparatus 100 and control signals are communicated wirelessly from the control module to treatment panels 120.

In some embodiments, the control module controls treatment parameters of ultrasonic energy and/or heat removal independently for each treatment panel 120. Ultrasonic energy parameters comprise ultrasonic intensity, frequency, and duty cycle. Heat removal parameters comprise surface temperature. By independent control of treatment parameters of each treatment panel, treatment may thereby be customized locally to different portions of the treatment volume underneath each treatment panel, depending on treatment requirements of each portion.

FIGS. 1B-1C show examples of treatment panels 120 arranged in two dimensions. In some embodiments, treatment panels 120 of an apparatus 100 may have non-uniform sizes, a combination of different shapes, and/or may be arranged in a pattern other than those shown, according to anatomical and treatment requirements. Treatment panels 120 may provide coverage over the entire area of the inside surface of sleeve 110, or may be limited to a specific area, as shown in FIG. 1D.

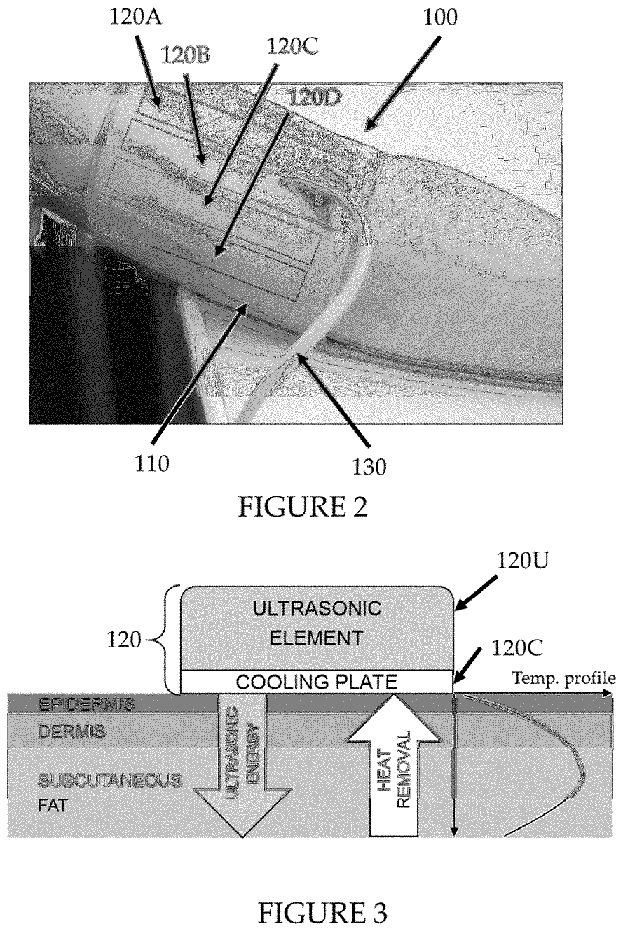

FIG. 2 illustrates a patient-attached apparatus 100 with four treatment panels 120A-120D inside of a sleeve 110 fixedly tightened around an upper arm of a patient.

In some embodiments, patient-attached apparatus 100 further comprises one or more types of light-emitting therapy devices, such as low-level laser therapy (LLLT), laser skin resurfacing, LED light therapy, and combinations thereof. Preferably, these additional elements are also fixedly arranged on the inside wall of sleeve 110.

Reference is now made to FIG. 3, illustrating the thermal effect of a treatment panel 120 on a treatment volume. Ultrasound element 120U emits ultrasonic energy into the surface of the treatment volume. The ultrasonic energy propagates into the treatment volume and produces heat that provides the treatment. Cooling plate 120C removes heat from the surface of the treatment volume, enabling ultrasound element 120U to operate at higher intensity—thereby generating heat in layers of superficial fascia and other treated subcutaneous fat tissue—without overheating the epidermis or dermis. Stronger cooling (i.e., a lower skin surface temperature) by cooling plate 120C permits ultrasound elements 120U to emit stronger ultrasound reaching deeper into treatment volume. Ultrasonic element 120U and cooling plate 120C may thereby be both controlled so as to select an ultrasonic energy intensity and heating penetration depth.

Besides ultrasound intensity, frequency of ultrasound emitted by ultrasonic element 120U may be varied to affect treatment depth. For example, lower ultrasonic frequencies can be used for deeper treatment and higher ultrasonic frequencies for shallower treatment; because the higher frequencies undergo higher attenuation and are converted to heat more superficially than the lower frequencies. Additionally, duty cycle of the ultrasonic energy may be varied; i.e., reduced to prevent heat build-up in portions of the treatment volume requiring high-intensity ultrasound treatment.

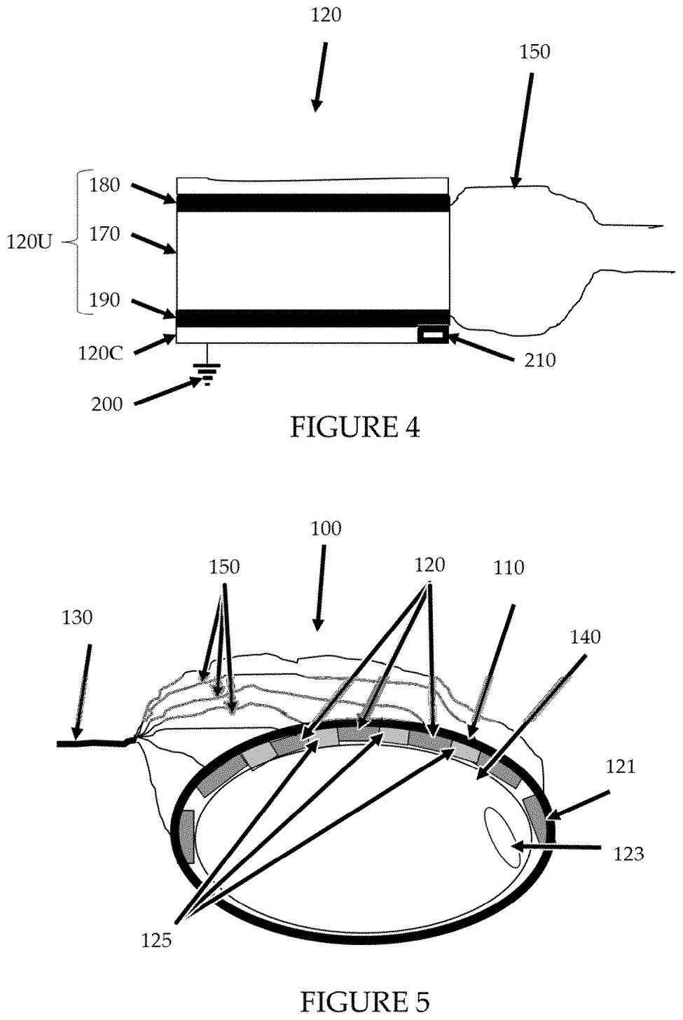

Reference is now made to FIG. 4, illustrating construction of a treatment panel 120, according to some embodiments of the invention.

Ultrasonic element 120U is composed of a piezoelectric layer 170. Piezoelectric layer 170 is composed of a piezoelectric material such as lead zirconate titanate (PZT) or a composite material. Ultrasonic element 120U further comprises two conductive plates 180 and 190. Conductive plates 180 and 190 are connected through wires 150 to an alternating-current power source, which can be disposed in a control module (not shown). One of the conductive plates 190 is connected to electrical ground 200. To meet regulatory safety requirements, grounded conductive plate 190 is usually in electrical connectivity with the body.

Conductive plate 190 is connected to a cooling device 120C, in order to keep the skin surface temperature at a requisite temperature. The requisite temperature is below a level that damages the skin. Typically, the requisite temperature is less than about 40° C. Adjacent to conductive plate 190 there may be one or more temperature sensors 210. Alternatively, or in addition, temperature sensor(s) 210 may be located within cooling device 210. Each temperature sensor 210 is wired to the control module through tether 130. Temperature sensor 210 monitors the skin temperature, by measuring either heat conduction or infrared radiation from the skin surface. Readings of temperature from temperature sensor 210 enable closed-loop control of ultrasonic element 120U and cooling plate 120C, in order to maintain the requisite skin temperature.

Each treatment panel's 120 parameters of ultrasound (intensity, frequency, and duty cycle) and of cooling (skin surface temperature) may be independently controlled, including stopping of treatment altogether. Additionally, the parameters may be time-varied, either in predetermined temporal profiles or in response to feedbacks measured during a treatment.

Reference is now made to FIG. 5, illustrating a patient-attached apparatus 100 with a sleeve 110 wrapped around a limb 140 of a patient. A treatment volume portion 123 is being treated by one 121 of treatment panels 120. In some embodiments, ultrasound imaging transducers 125 are interspersed among treatment panels 120, as shown. Alternatively, or in addition, some or all of the ultrasonic elements 120U of treatment panels 120 may also function as ultrasound imaging transducers 125. Ultrasound imaging transducers 125 enable acquisition of ultrasound images of the treatment volume. The images may be acquired before and/or after the treatment, as well as in real time during the treatment. In-treatment images can be analyzed to determine cumulative effects of the treatment at varying depths in real time, enabling a closed-loop feedback for control of treatment panels 120. The treatment may therefore be adapted to real-time conditions of the treated volume.

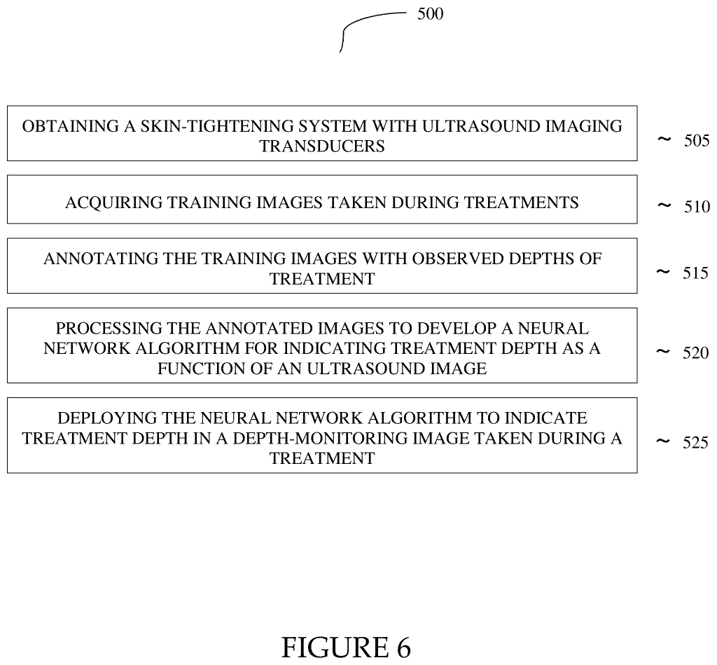

Reference is now made to FIG. 6, showing steps of a method 500 for providing real-time in-treatment depth indications from ultrasound images of a treatment volume, according to some embodiments of the invention.

After obtaining a system of the invention 505, including ultrasound imaging capability, an aggregation of ultrasound training images are acquired during treatments 510. The training images are annotated (e.g., by an ultrasound specialist) with observed depths of treatment 515. The aggregation of annotated training images is processed by a neural network algorithm for indicating treatment depth as a function of an ultrasound image 520. The neural network algorithm is then deployed to indicate treatment depth of a depth-monitoring image during a treatment. The depth-monitoring image and/or the indicated depth may be displayed, so that a technician can visualize the treatment in real time. Additionally, the indicated depth may be fed back to the control module in order to adjust treatment parameters in real time.

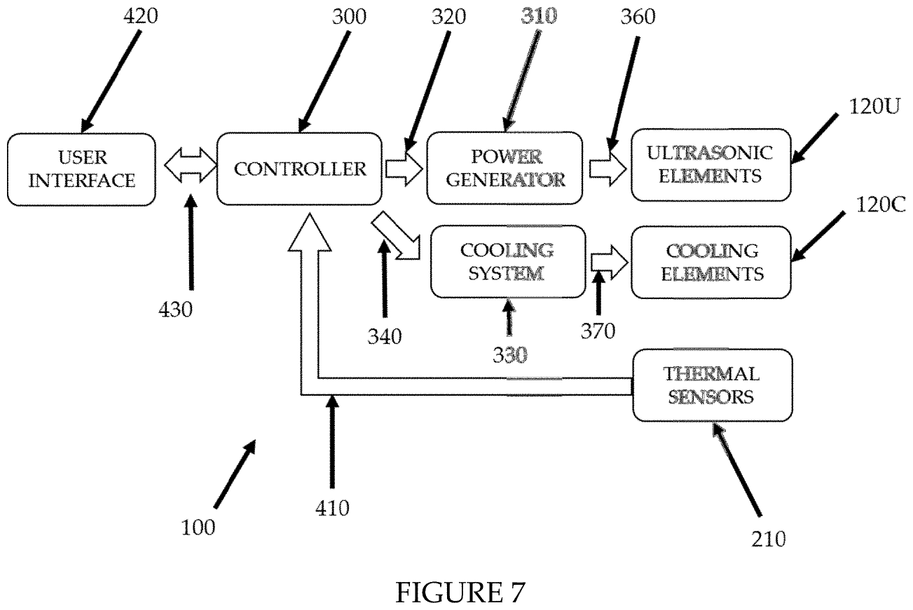

Reference is now made to FIG. 7, showing functional block diagrams of a skin tightening system 101 according to some embodiments of the invention. A control module 300 computes ultrasound parameters (intensity, frequency, and duty cycle) and skin surface temperature required to achieve requisite treatment by each treatment plate 120 to a treatment volume portion underneath the treatment plate 120. Control module may employ temperature feedback 410 of thermal sensors' 210 outputs to regulate the temperature. A power generator 310 receives the computed ultrasound parameters from control module 300 and drives ultrasonic elements 120U accordingly. A cooling system 330 receives the computed skin surface temperature from control module 300 and drives cooling elements 120C accordingly. A user interface 420 allows a user to set treatment parameters and monitor progress of treatment.

In some embodiments, control module 300 is pre-programmed to follow a particular regimen of ultrasound and cooling parameters. In some embodiments, control module 300 may receive instructions from a user through a user interface 420 (including a display) of the control module 300. Using the user interface 420, the user may set which ultrasonic elements are to be used, their activation times, and the skin surface temperature needed. Alternatively, the user may adjust frequency, power and duty cycle of each of the ultrasonic elements 120U. Through the interface 420 the user may also receive an indication of the skin temperature measured by thermal sensors 210.

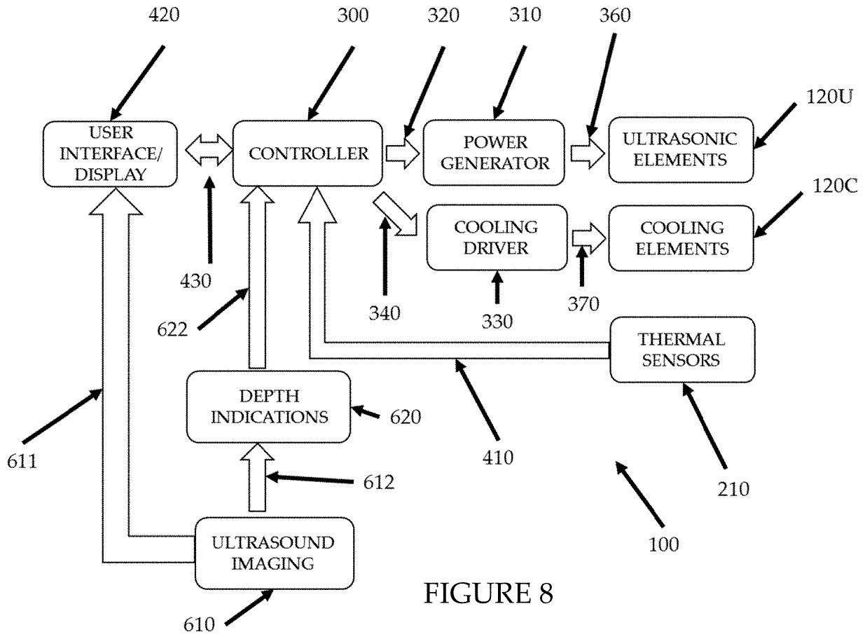

Reference is now made to FIG. 8, showing a functional block diagram of a skin tightening system 102 with ultrasound imaging feedback, according to some embodiments of the invention. Ultrasound imaging system 610 (comprising ultrasound imaging transducers; see FIG. 5) captures ultrasound images during imaging. Depth indications 620 are extracted from the images. In some embodiments, depth indications 620 are extracted from the image by segmentation of the different layers in many ultrasound images and learning by a neural network. During the course of a treatment, control module 300 receives feedback 622 of depth indications 620, specifying a measured depth of the superficial fascia. Control module 300 employs depth indications 620 to compute adjusted parameters of treatment, including updated ultrasound and cooling parameters. User interface 420 may display the images, so that a technician can visualize the treatment in real time. User interface 420 may display depth indications 620.

### Experimental Results

Experimental studies and results supporting operability of the invention are now described.

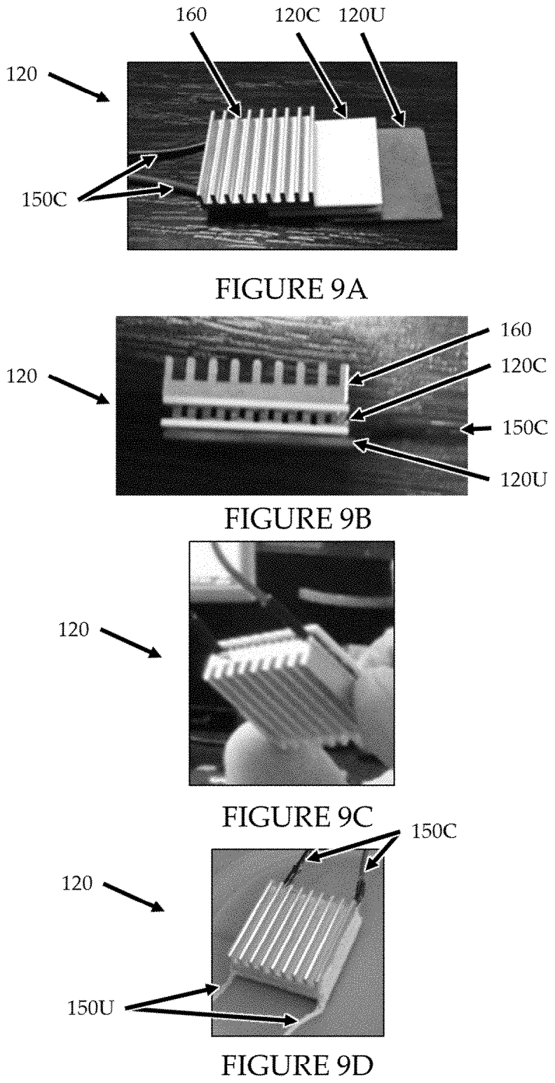

FIGS. 9A-9D show a prototype treatment panel 120 at various stages of assembly. Ultrasound element 120U comprises a piezoceramic plate (ferroelectrically hard PZT type 2.3 MHz 1^{st} harmonic frequency), with electrical lead wires 150U. Cooling element 120C comprises a Peltier cooler, with electrical lead wires 150C. Treatment panel further comprises a heat exchanger 160 (in the embodiment shown, a plate-fin heat exchanger) for dissipating heat from cooling element 120C.

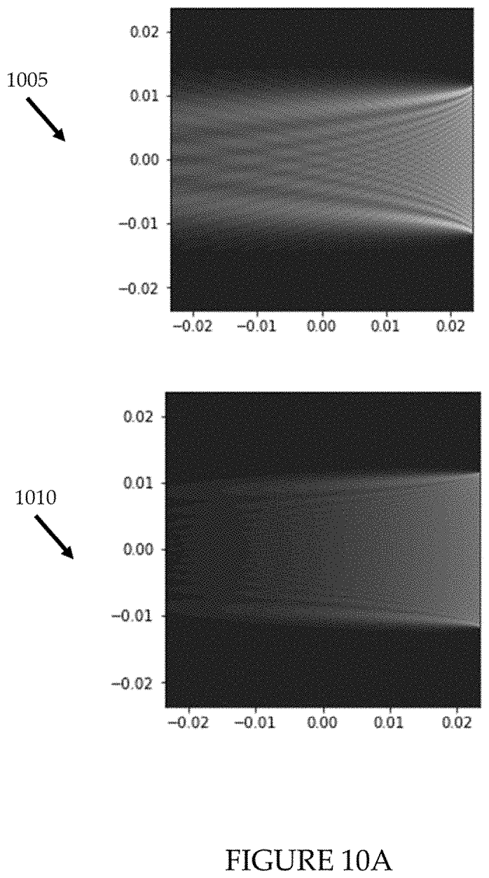

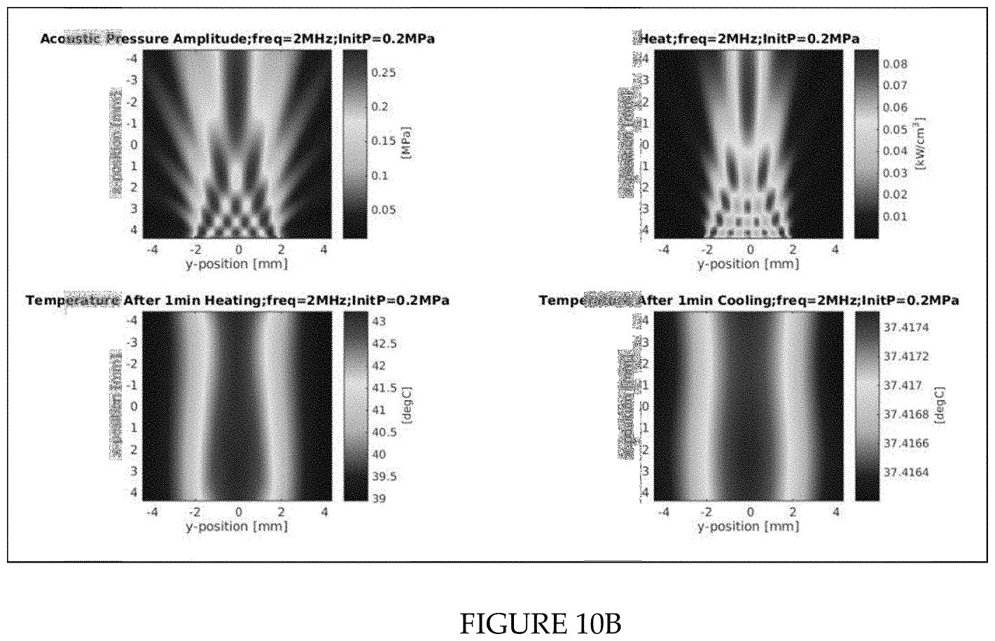

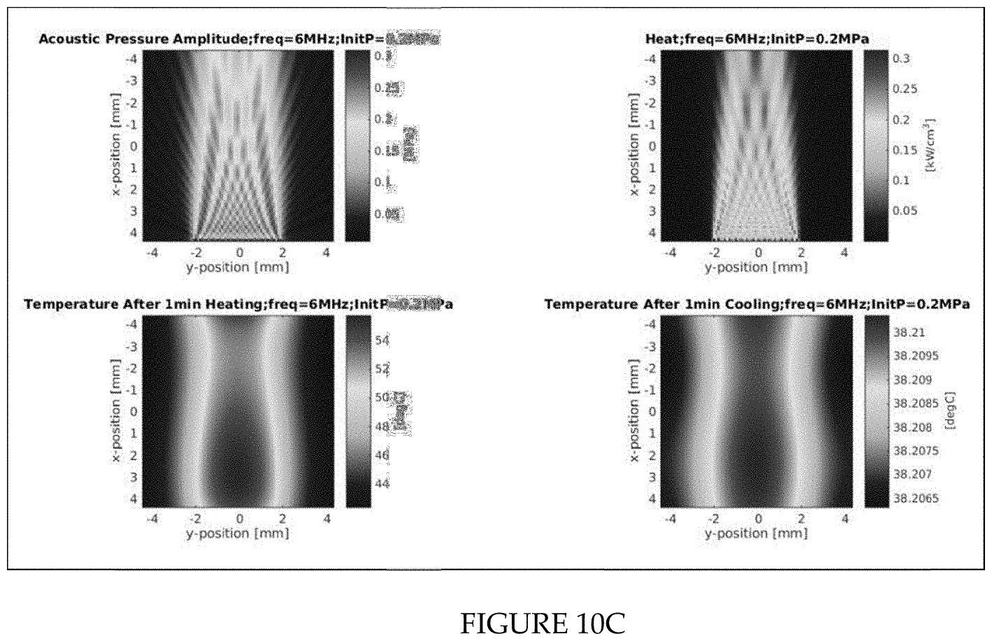

FIGS. 10A-D show the results of theoretical calculation and modeling of tissue heating by ultrasound. Modeling techniques employed included finite-difference time domain (FDTD), k-wave, and finite elements.

FIG. 10A shows acoustic pressure from acoustic waves in tissue at 2 MHz 1005 and at 6 MHz 1010, without thermal conductivity and skin cooling. (Distances are in meters).

FIGS. 10B and 10C show, for 2 MHz and 6 MHz respectively, profiles of acoustic pressure, heat generation, temperature after 1 minute of heating, and temperature after 1 minute of cooling.

FIG. 10D shows examples temperature layer profiles of tissue heating with the Peltier element holding skin temperature at 25° C. At 2 MHz ultrasound frequency 1020, heating time to 48° C. is 235 seconds and the peak temperature is 10 mm from the skin surface. At 6 MHz ultrasound frequency 1025, heating time to 48° C. is 77 seconds and the peak temperature is 5 mm from the skin surface.

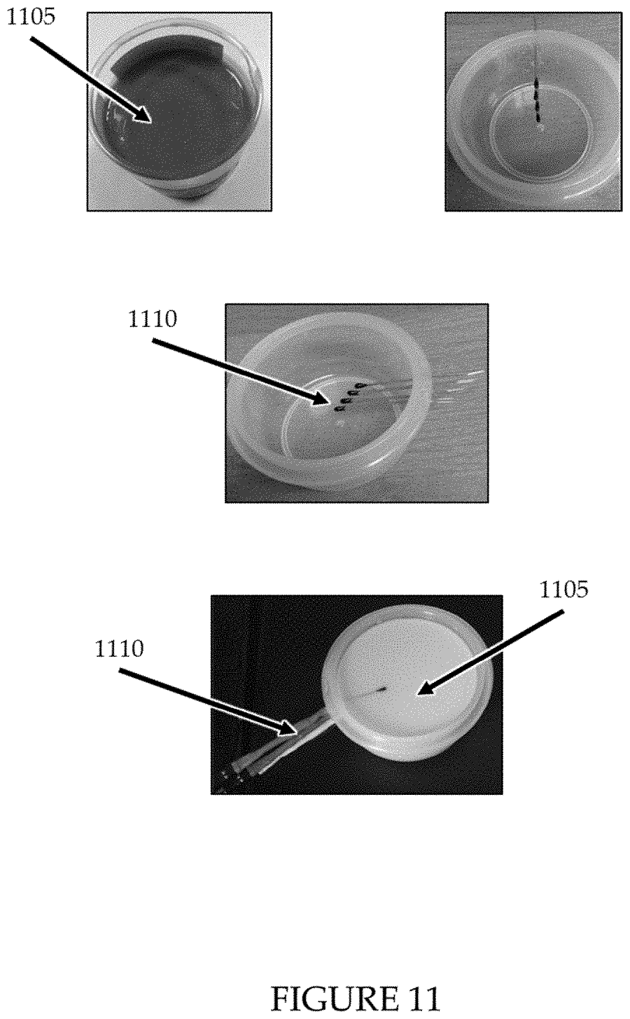

FIG. 11 shows creation of ultrasonic imaging phantoms. Materials used included polyurethane and silicone rubber. Thermistors (Epcos S861, 10 KOM, 1%, NTC) were placed at surface layer and at 5, 10, and 15 mm below the surface.

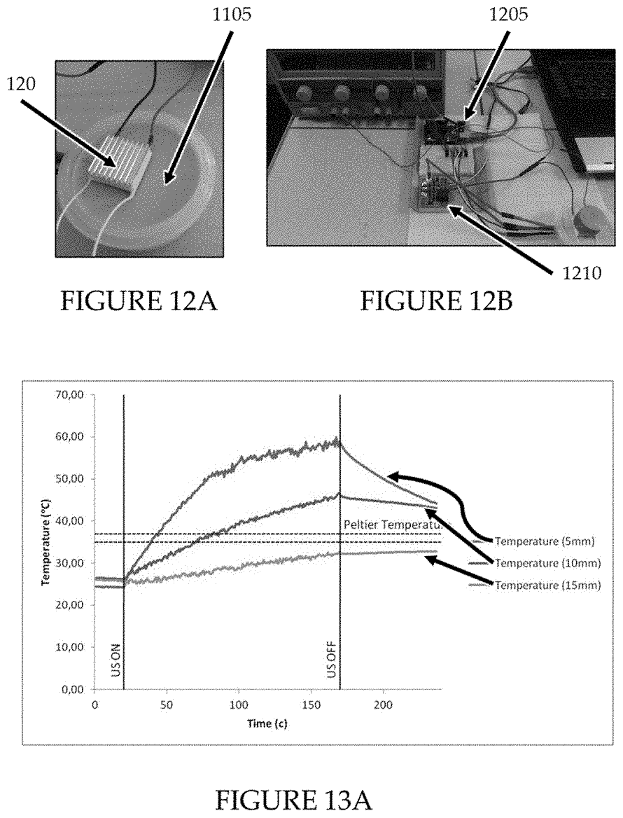

FIG. 12A shows prototype treatment panel 120 on the imaging phantom 1105. FIG. 12B shows the test setup, which includes a microcontroller board 1205 for thermistor data collection (Arduino UNO) and a controller board 1210 for Peltier element control.

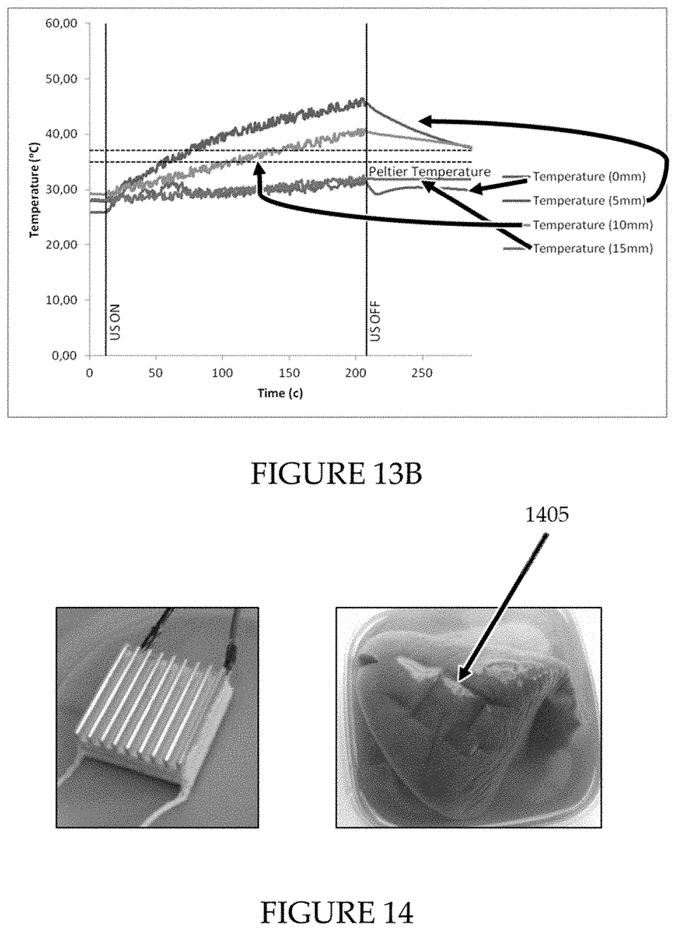

FIGS. 13A-13B show temporal heating of the imaging phantom for 2.3 MHz ultrasound, CW, at layers 5-15 mm deep. FIG. 13A is for electric power of 10 W, acoustic power of 5 W, and power flux density 1 W/cm²; FIG. 13B is for electric power of 6 W, total acoustic power 3 W, power flux density 0.8 W/cm². In both figures, initial surface temperature was 26° C. Peltier was adjusted to hold 35° C., so it started cooling when the temperature on the skin surface exceeded 35° C. and held it constant. The results demonstrate the ability control the heating temperature of an internal layer of the imaging phantom (peaking at about 5 mm deep) while holding the surface layer at a lower, constant temperature.

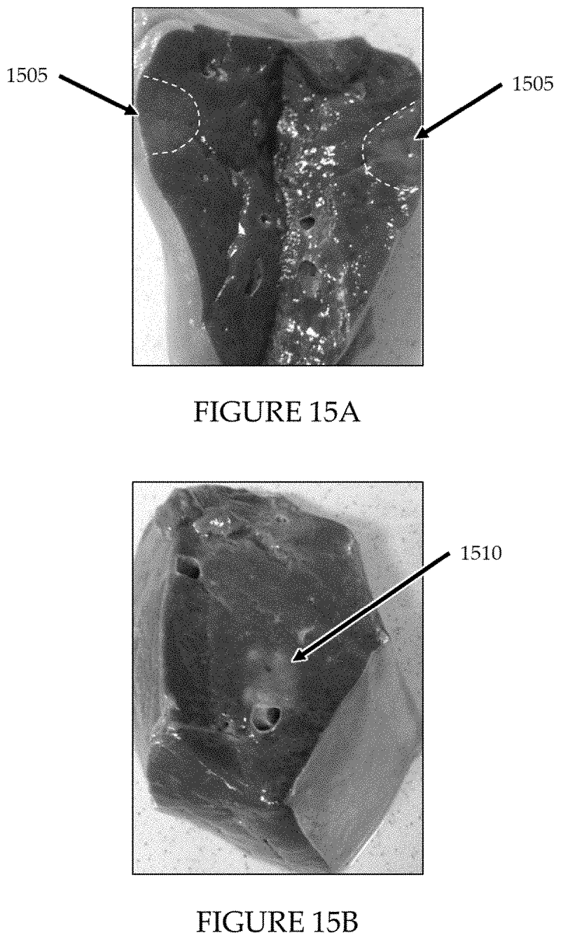

The prototype module 120 was tested on fresh bovine liver. FIG. 14 shows the prototype treatment module 120 and its imprint 1405 on the treatment area of a section of liver. The liver, in contact with the module 120, was subjected to total acoustic power of 10 W (power density 2.5 W/cm²), at a frequency of 2.3 MHz CW ultrasound, Peltier temperature 35° C. for a treatment time of 180 seconds. FIG. 15A shows a cross section of the treatment volume after treatment. Note the regions of light coagulation 1505 under the treated surface. While the surface of the treatment volume was undamaged, FIG. 15B shows strong burning 1510 on the side of the liver section opposite the treatment surface, at a depth of 4 mm below the treatment surface.

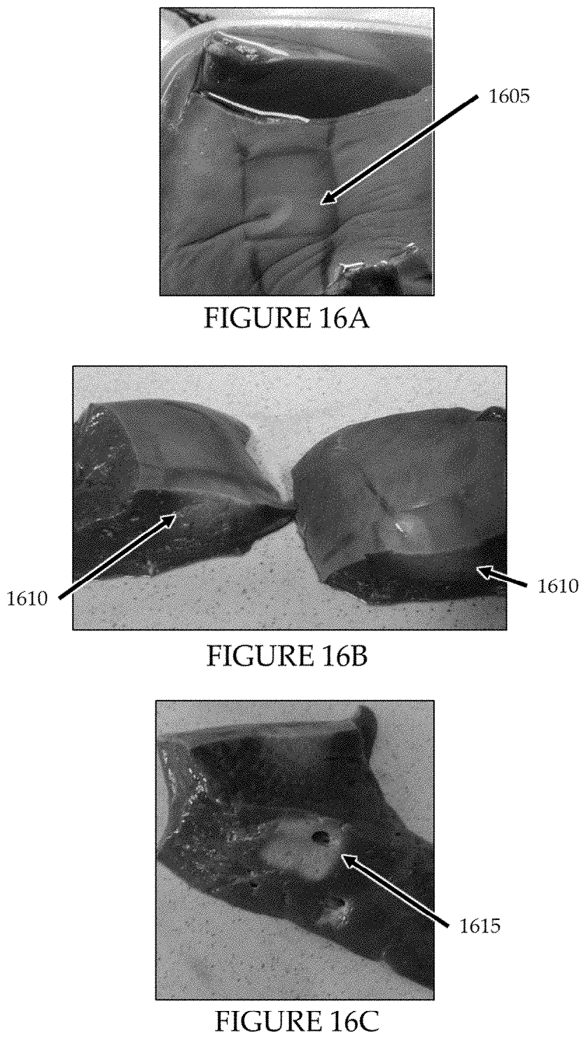

FIG. 16A shows a liver treatment volume subjected to total acoustic power of 22 W (power density 5 W/cm²), at a frequency of 2.3 MHz CW ultrasound, Peltier temperature 55° C. for a treatment time of 300 seconds. Note the strong burning 1605. FIG. 16B shows coagulation 1610 under the surface. FIG. 16C shows strong burning 1615 on the opposite side, at 4 mm depth.

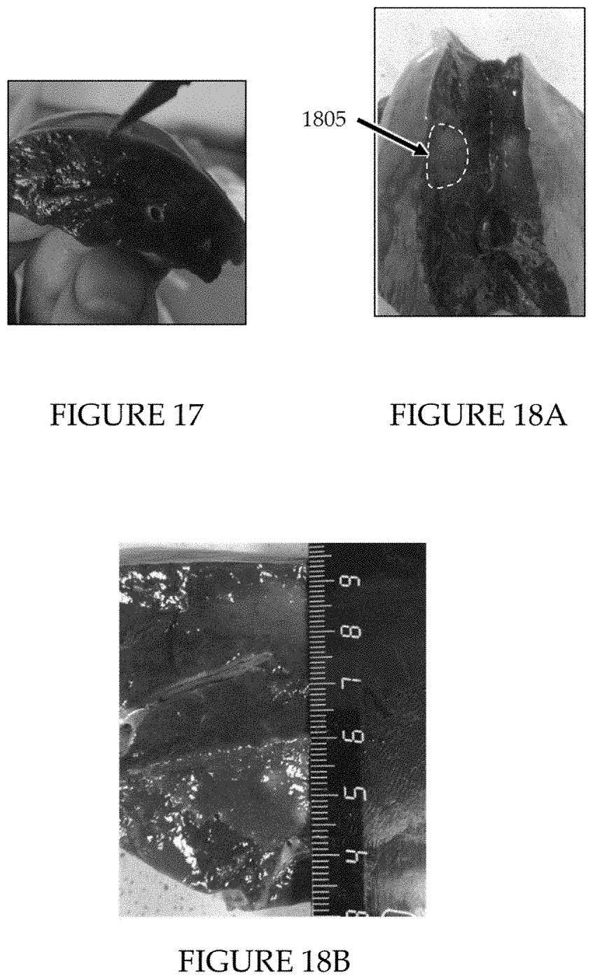

FIG. 17 shows a liver treatment volume subjected to total acoustic power of 5 W (power density 1 W/cm²), at a frequency of 2.3 MHz CW ultrasound. Peltier temperature 35° C. for a treatment time of 180 seconds. There is light coagulation extending 2 mm below the surface and burning on the backside of liver piece is absent. This is in comparison to the results shown in FIGS. 15A-B, which occurred under twice the acoustic power and for a 67% longer treatment time.

FIG. 18 shows a liver treatment volume subjected to total acoustic power of 7 W (power density 2 W/cm²), at third-harmonic frequency of 6.8 MHz CW ultrasound. Peltier temperature 35° C. for a treatment time of 180 seconds. Note the region of liver ablation 1805 under the surface. As shown in FIG. 18B, the treatment volume for 6.8 MHz ultrasound is deeper (starting about 4 mm below the surface) and thicker (about 1 cm thick) than the superficial 2-mm thick light coagulation shown in FIG. 17, under 2.3 MHz ultrasound. It is expected that the extent of coagulation at 6.8 MHz may be reduced and adjusted by applying the ultrasound at selected reduced duty cycles.

1. A system for providing an ultrasonic skin-tightening or body-shaping treatment, comprising
a. a sleeve configured for fixedly wrapping around an organ of a mammalian body; said organ comprising a treated volume of a layer of tissue(s) underneath an area of skin surface of said organ;
b. an arrangement of one or more treatment panels disposed on an inside surface of said sleeve, each said treatment panel comprising
i. an ultrasonic element, configured to provide ultrasound waves to a portion of said treated volume underneath said treatment panel;
ii. a cooling plate, configured to remove heat from said treated volume portion; and
c. a control module, in electrical connection with said treatment panels, configured to receive temperature outputs of said temperature sensors and to control said ultrasonic elements—including intensity, frequency, and/or duty cycle of said ultrasound waves—and to control said cooling plate temperature;
wherein said control module is further configured to control said ultrasonic elements and said cooling plates of each said treatment panel independently, thereby enabling hands-free treatment, with localized variations in said controls as needed, throughout said treatment volume.

2. The system of claim 1, wherein one or more of said treatment panels comprises a temperature sensor configured to monitor temperature on an outside surface of said treated volume portion.

3. The system of claim 1, wherein said controller is further configured to receive one or more of preliminary inputs from the user according to anatomical area and size of patient.

4. The system of claim 1, wherein said sleeve comprises a wrap-around cuff or a closed elastic loop.

5. The system of claim 1, wherein said sleeve is configured for wrapping around one or more of an arm, the neck, the abdomen, the back, a thigh, and the face.

6. The system of claim 1, wherein said treatment panels are arranged on said sleeve inside surface in a one dimension, two-dimensions, or any combination thereof.

7. The system of claim 1, wherein said treatment panels cover a portion of skin underneath said sleeve.

8. The system of claim 1, wherein said control module is configurable to disable one or more of said treatment panels during any time interval of said treatment.

9. The system of claim 1, wherein said control module is further configured to change of ultrasound parameters to change from skin tightening to fat destruction.

10. The system of claim 1, further comprising a tether providing electrical connections to said ultrasonic elements and said cooling plate from said control module.

11. The system of claim 1, wherein depths below skin surface of a layer of said treatment, in a portion of said treatment volume underneath one or more of said treatment panels, is controlled by varying one or more of said intensity, frequency, and temperature.

12. The system of claim 11, wherein said system is configured for treatment of skin laxity—wherein said depths are in a range of about 3-20 mm below the skin surface; and of fat deposits—wherein said depths are in a range of about 10-30 mm below the skin surface.

13. The system of claim 1, further comprising treatment elements providing low-level laser therapy (LLLT), laser skin resurfacing, LED light therapy, any other light-emitting therapy device, or any combination thereof.

14. The system of claim 1, further comprising ultrasound imaging transducers, said system further configured to
a. acquire real-time ultrasound images of said treatment volume before, during, and/or after said treatment;
b. analyze said images during treatment to determine cumulative effects of said treatment at varying depths of said treatment volume; and
c. adjust said treatment parameters in real time as a function of said cumulative effects.

15. The system of claim 14, wherein said control module is configured to employ a neural network algorithm to compute said depth indication.

16. The system of claim 15, wherein said system is further configured to determine a depth of treatment prior to the treatment.

17. The system of claim 15, wherein said control module comprises a user interface that displays said real-time image and/or said depth indication.

18. A method for providing real-time in-treatment depth indications from ultrasound images of a treatment volume, comprising steps of
a. obtaining the system of claim 14;
b. acquiring training images taken during treatments by said system;
c. annotating the training images with observed depths of treatment;
d. processing an aggregation of said annotated images to develop a neural network algorithm for indicating treatment depth as a function of an ultrasound image;
e. deploying the neural network algorithm to indicate a treatment depth in a depth-monitoring image taken during a treatment.
